(19) United States
(12) Patent Application Publication
(10) Pub. No.: US 2004/0101563 A1
(43) Pub. Date: May 27, 2004

(54) Storage stable antihistaminic syrup formulations

(76) Inventors: Subhas C. Kundu, Tappan, NY (US); Ed Koch, New Freedom, PA (US); Robert T. Tambe, Baltimore, MD (US)

Correspondence Address: DICKSTEIN SHAPIRO MORIN & OSHINSKY LLP, 2101 L STREET NW, WASHINGTON, DC, 20037-1526, US

(21) Appl. No.: 10/621,316

(22) Filed: Jul. 18, 2003

Related U.S. Application Data
(60) Provisional application No. 60/396,566, filed on Jul. 18, 2002.

(51) Int. Cl. A61K 9/14

(52) U.S. Cl. 424/488

(57) ABSTRACT

Storage stable, pleasant tasting oral antihistaminic syrup compositions free of sugars and aminopolycarboxylic acids, and methods of treating allergic reactions, mental disorders, and/or vascular disorders in a mammal by administering such compositions.

### CROSS REFERENCE TO RELATED APPLICATIONS

This application claims the priority of provisional application serial No. 60/396,566, filed Jul. 18, 2002, which is hereby incorporated by reference in its entirety.

### FIELD OF THE INVENTION

The field of the this invention is oral syrup antihistaminic pharmaceutical formulations and their use in treating allergic conditions, mental disorders, and vascular disorders.

### BACKGROUND OF THE INVENTION

Pharmaceutical formulations containing antihistamines are indicated for the treatment of various allergic conditions. Oral solutions containing pharmaceutical agents, such as antihistamines, are sometimes preferred to tablets or other dosage forms, particularly, for example, when administration to elderly or pediatric patients is desired. Such oral formulations traditionally are concentrated sugar solutions or syrups. U.S. Pat. No. 4,282,233 refers to loratadine and a syrup formulation comprising an antihistamine, together with sucrose, sorbitol, propylene glycol, methylparaben, propylparaben, color, alcohol, flavor, and water. Syrup formulations containing sugar and related piperdine antihistamines have been marketed as, for example, azatadine oral syrup (Zadine® Schering), loratadine oral syrup (Claritin® Schering), and cyproheptadine (Periactin® Merck).

An important goal in formulating liquid pharmaceutical compositions is minimizing the degradation of the active ingredients. To that end, nitrogen is sometimes used during manufacture and in the headspace of packaging to enhance the stability of the active pharmaceutical ingredient.

U.S. Pat. No. 6,132,758 (“the '758 patent”) indicates that a currently marketed syrup formulation containing an antihistamine (loratadine), together with citric acid, an artificial flavor, glycerin, propylene glycol, sodium benzoate, sucrose, and water, generates degradation products under certain storage conditions involving contact with the air. According to the '758 patent, oral antihistaminic syrup formulations can be stabilized against degradation by including in the formulations an aminopolycarboxylic acid, such as ethylenediaminetetraacetic acid (EDTA). The '758 patent provides a comparison of formulations with EDTA and without EDTA and concludes that EDTA significantly inhibits the degradation of loratadine.

U.S. Pat. No. 6,472,401 (“the '401 patent”) refers to the use of antihistaminic compounds to treat mental or vascular disorders in patients. According to the '401 patent, there is a correlation between increased allergic conditions and mental disorders. Mental disorders, such as aggression and depression, lead to lower levels of 5-hydroxyindoleacetic (5-HIAA), which is the primary metabolite of serotonin (5-HT). Abnormal 5-HT function is associated with mental disorders. In fact, serotonin antagonists and agonists are commonly used drugs for treatment of neuropsychiatric disorders (e.g., buspirone, clozapine). According to the '401 patent, 5-HT, which is present in vascular tissue, is also associated with vascular-associated disorders such as migraines, stroke, orthostatic hypotension, gastrointestinal stasis, nausea, dizziness, and jet lag.

What is needed are stable antihistaminic syrup formulations for the treatment of allergic conditions, mental disorders, and vascular disorders without the addition of an aminocarboxylic acid to achieve the desired stability.

### SUMMARY OF THE INVENTION

We have now unexpectedly and surprisingly discovered that by removing sugars from the prior art antihistaminic syrup formulations, superior storage stability can be achieved without the addition of any aminopolycarboxylic acid. Thus, in contrast to the teachings of the '758 patent, we have discovered that it is not necessary to include an aminopolycarboxylic acid in an antihistaminic formulation of loratadine and related antihistaminic compounds in order to achieve a pleasant tasting syrup having significantly enhanced storage stability in terms of reduction of degradation products.

Degradation of previous antihistaminic syrups is observed during storage stability testing as evidenced by declining concentrations of the active ingredient and a concomitant formation of degradation products over time. Two degradation products which typically form in conventional loratadine syrup formulations have been identified as 2-Hydroxymethyl loratadine (“2-HML”) and 4-Hydroxymethyl loratadine (“4-HML”). 2-HML and 4-HML degradation products are also generated in other piperidine antihistamines when a hydroxymethyl group attaches to the pyridine ring during storage of the product. Other degradation products also typically occur.

The present invention provides a storage stable, or essentially sugar-free, oral pharmaceutical composition, comprising a therapeutically effective amount of a piperidine antihistamine, a viscosity imparting agent, a preservative, a buffer to control pH to about 2 to about 4, and water, wherein the composition does not contain an aminopolycarboxylic acid. The present invention also provides methods for treating an allergic condition, mental condition, or vascular condition, by administering a storage stable sugar-free pharmaceutical composition which is an aqueous solution comprising a therapeutically effective amount of an antihistamine, wherein the composition does not contain an aminopolycarboxylic acid.

The present invention solves the problem of antihistamine degradation in oral solutions for the treatment of allergic conditions, mental disorders, and vascular disorders and does so without resorting to the inclusion of an aminopolycarboxylic acid, such as EDTA. Thus, the present invention provides numerous advantages over the prior art. Moreover, if desired, no nitrogen is needed in the manufacture or packaging of the pharmaceutical composition of the present invention. The use of nitrogen renders such processes more difficult, and requires careful control to assure that both the product and workers are adequately protected from excessive use of nitrogen.

Additional features and advantages of the invention will be set forth in the description which follows and will be apparent from the description or may be learned by practice of the invention.

### DETAILED DESCRIPTION OF THE INVENTION

Reference will now be made in detail to the presently preferred embodiments of the invention, which, together with the following examples, serve to explain the principles of the invention. It is to be understood that the application of the teachings of the present invention to a specific problem or environment will be within the capabilities of one having ordinary skill in the art in light of the teachings contained herein. Examples of the products of the present invention and processes for their preparation and use appear in the following examples.

The present invention provides storage stable, essentially sugar-free pharmaceutical compositions which are aqueous solutions comprising a therapeutically effective amount of a piperidine antihistamine. The compositions do not resort to use of an aminopolycarboxylic acid, but instead achieve significantly enhanced stability in terms of reduction of degradation products by providing essentially sugar-free syrup formulations utilizing a viscosity enhancing agent and one or more non-sugar sweeteners. Suitable antihistamines include, but are not limited to, loratadine, azatadine, descarboethoxyloratadine, ketotifen, astemizole, terfenidine, fexofenadine, and cyproteptadine. In a preferred embodiment, the antihistamine is loratadine, descarboethoxyloratadine, or azatadine.

The formulations according to the invention may also contain additional active pharmaceutical ingredients, such as, for example, decongestants, analgesics, antitussives, and expectorants. Any specific drugs within these therapeutic classes are suitable for inclusion in the formulations of the invention. Illustrative examples include analgesics such as aspirin, acetaminophen, naproxen, ketoprofen, and ibuprofen; decongestants such as pseudophedrine or phenylpropanolamine; antitussives such as codeine, hydrocodone, or dextromethorphan; and expectorants such as guaifenesin, including salts thereof.

Under stability analyses the storage stable compositions of the present invention show remarkable reductions in degradation products and increases in potency as compared to previous liquid antihistaminic formulations. After 8 to 12 weeks at 60° C., the formulations according to the invention exhibit degradation products present in an amount of 0.1% or less as a percentage of the active ingredient.

As used herein, “storage stable” means liquid pharmaceutical formulations containing an active antihistaminic compound and in which degradation products which are typically observed in storage stability testing of such formulations are absent or significantly reduced during storage stability testing. The aminopolycarboxylic acid-containing formulations referred to in the '758 patent yield degradation product levels of up to 0.62% when tested under severe storage conditions, i.e., 55° C. for up to 12 weeks. The formulations of the present invention, which do not contain an aminopolycarboxylic acid, are at least as storage stable as the formulations referred to in the '758 patent and, more preferably, yield degradation product levels of less than about 0.1% of the level of the active ingredient when tested at 60° C. for 8 weeks.

The compositions of the invention are pleasant tasting and sugar free or essentially sugar free so as to achieve significantly-enhanced storage stability. By essentially sugar-free, we mean that the formulation can include no sugar or no more sugar than is necessary to achieve storage stable formulation without use of an aminopolycarboxylic acid. Various non-sugar sweeteners are suitable for use in the formulations of the present invention. Examples include polyols such as maltitol and sorbitol, saccharin, aspartame, sodium cyclamate, calcium cyclamate, sucralose, and acesulfame K. A preferred sweetener is sodium saccharin.

Formulations according to the invention for oral administration may include various excipients, for example, sweeteners, thickeners, buffering agents, preservatives, flavorants, and solubilizers.

In the absence of sugar, it may be desirable in certain applications to include various thickening or viscosity-increasing agents to bring about a syrupy consistency to the formulation. For example, hydroxyethyl cellulose, methyl cellulose, sodium carboxymethyl cellulose, microcrystalline cellulose, hydroxypropyl cellulose, hydroxypropyl methylcellulose, other cellulose derivatives, gelatin, polyethylene glycol, and water-soluble carboxyvinyl polymers can be used. In one embodiment, the viscosity-increasing agent is hydroxypropyl methylcellulose, although any other suitable thickening agent can be used, in an amount sufficient to raise the viscosity of the formulation to above the viscosity of water at the same temperature.

The formulation also may contain an antimicrobial component or agent to ensure safe storage without the proliferation of pathogenic molds, yeasts, or bacteria. Various antimicrobials which are suitable for use in foods and other ingestable substances are known in the art and can be used in the present invention. Examples include the parabens (butylparaben, methylparaben, and propylparaben), propyl-p-hydroxybenzoates, sodium benzoate, and sorbic acid. A preferred antimicrobial agent is sodium benzoate.

Various buffers and buffer salts used to maintain pH also are suitable for use in the present invention. The formulations according to the invention will typically have a pH of about 2 to 4. Examples of buffers include tartaric acid, maelic acid, phosphoric acid and citric acid.

In preparing the formulations of the present invention, the active antihistaminic component is preferably incorporated into an aqueous-based carrier. In addition to water, the formulations may also contain one or more co-solvents to assist in solubilization and incorporation of water-insoluble components. Various co-solvents are suitable for use in the present invention. For example, propylene glycol, sorbitol solution, glycerin, polyethylene glycol, and ethyl alcohol can be used.

Various flavors or flavoring agents may be included to impart a pleasant taste. A pleasant taste is particularly important when the formulation is intended for administration to children. Numerous flavors that are commonly used in pharmaceuticals, foods, candies and beverages are also suitable for use in the present invention. Examples include fruit, peppermint, licorice, bubble gum, and other flavors.

The formulations of the present invention can be prepared by various methods. One embodiment of a manufacturing method is as follows.

### Manufacture of an Essentially Sugar-Free Antihistaminic Liquid Pharmaceutical Formulation

### Formula

| | | Quantity | |
|---|---|---|---|
| Item# | | per 100 mL | |
| 1 | Purified Water, USP | 15.5 | mL |
| 2 | Hydroxypropyl Methylcellulose, 2910, USP | 0.450 | g |
| 3 | Purified Water, USP | 60.5 | mL |
| 4 | Sodium Saccharin, USP | as required | |
| 5 | Sodium Benzoate, NF | 0.100 | g |
| 6 | Citric Acid, anhydrous, USP | 0.850 | g |
| 7 | Glycerin, 96%, USP | 9.00 | mL |
| 8 | Flavor(s) | as required | |
| 9 | Propylene Glycol, USP | 9.10 | mL |
| 10 | Loratadine | 0.100 | g |

| | | |
|---|---|---|
| 11 | Purified Water, USP | q.s. ad 100 mL |

### Process

Step

A Transfer hot (80 to 85° C.) purified water, USP (item #1) into a suitable container. While mixing, add and disperse hydroxypropyl methylcellulose, 2910, USP (item #2).

B Transfer purified water, USP (item #3) into a suitable container. Add and dissolve the following ingredients:

sodium saccharin, USP (item #4)

sodium benzoate, NF (item #5)

citric acid, anhydrous, USP (item #6)

glycerin, 96%, USP (item #7)

Transfer Step B into Step A while mixing. Continue mixing while cooling the batch to below 30° C.

C Add flavor(s) to the batch while mixing.

D Transfer propylene glycol, USP (item #9) into a suitable size container and heat to 40 to 45° C. Add loratadine (item #10) while mixing. Mix well until loratadine is dissolved. Transfer Step D into the batch while mixing.

E Adjust the batch volume to 100 mL with purified water, USP (item #11). Mix well until the batch is uniform.

Loratadine and related compounds are typically not water soluble. Thus, a solvent (e.g., propylene glycol) may be used. To effect the complete solution in a reasonable amount of time, the solvent may be warmed to about, for example, 40° C. Also in regard to the loratadine, the pH of the bulk should desireably be acidic (preferably around pH 3) prior to adding the loratadine. If the pH of the bulk is neutral or basic, the antihistamine active may precipitate. Thus, the addition of an acidifying agent (e.g., citric acid) may precede the addition of, for example, loratadine. Saccharin sodium and sodium benzoate may be added prior to acidifying the batch. In addition, sufficient solvent (e.g., water, propylene glycol, or glycerin) should be present to keep the acid forms of saccharin sodium and sodium benzoate in solution once the batch is acidified.

It is possible to hydrate the hydroxypropyl methylcellulose (HPMC) or other thickening agent using various methods. Instead of dissolving the sodium benzoate, sodium saccharin, citric acid, and glycerin in a separate portion of water, the water also may be added directly to the batch and then these items dissolved into the main batch. Another method of hydrating the HPMC is in cold water. In order to effect complete hydration without lumping, the HPMC may be first wetted in either, e.g., glycerin or propylene glycol. This slurry is then added to cold water and mixed until uniform. Another method is to use surface-treated HPMC. Surface treated HPMC can also be added directly to cold water. Once the HPMC is dispersed, the pH can be raised to above about pH 8. This allows the HPMC to hydrate. The pH can be raised, for example, by adding sodium saccharin and sodium benzoate to the batch. Once hydration is accomplished, the batch can be acidified with citric acid and other ingredients added.

After manufacture, the liquid antihistaminic formulations of the invention are packaged in any suitable container for use by health care providers and patients. Suitable packaging includes various glass, PET, and HDPE bottles, preferably opaque HDPE to further enhance long term stability.

The present invention also provides methods of treating allergic conditions in a subject. The methods include administering to a subject suffering from an allergic condition a storage stable pharmaceutical composition according to the invention. In one embodiment, the subject is human. In another embodiment, the allergic condition is seasonal allergic rhinitis or chronic idiopathic urticaria. The storage stable pharmaceutical composition can be administered to a patient in a dosage range of, for example, 0.5 mg to about 15 mg per day, preferably about 1 mg to about 12 mg per day, and more preferably 5 to 10 mg per day.

Further embodiments of the invention are directed to treatment of mental disorders and/or vascular disorders with antihistaminic syrup formulations. As described in U.S. Pat. No. 6,472,401, hereby incorporated by reference in its entirety, antihistaminic compounds can be used to treat mental disorders and/or vascular disorders. In particular, there is a high correlation between allergic disorders and mental disorders such as mood disorders and anxiety disorders (e.g., depression, alcoholism, weight management disorders, social disorder, impotent/sexual dysfunction, panic, and obsessive/compulsive disorder) leading to abnormal levels and function of serotonin (5-HT). Abnormal serotonin function is also associated with vascular disorders (e.g., migraines, stroke, orthostatic hypotension, gastrointestinal stasis, nausea, dizziness, and jet lag). The storage stable compositions of the present invention can be administered to a patient suffering from or susceptible to an allergic disorder, mental disorder, and/or vascular disorder or any combination of these disorders. In a preferred embodiment, the mental disorder is depression. The dose of the storage stable composition can vary depending on the individual patient and the severity of the mental disorder. In one embodiment, the storage stable compositions are administered to a patient in dosage ranging from in a dosage range of, for example, 0.5 mg to about 15 mg per day, preferably about 1 mg to 12 mg per day, and more preferably about 5 to 10 mg per day.

It is to be understood that application of the teachings of the present invention to a specific problem or environment will be within the capability of one having ordinary skill in the art in light of the teachings contained herein. The present invention is more fully illustrated by the following non-limiting examples.

### EXAMPLE 1

Studies were undertaken to determine the storage stability of a sucrose-containing loratadine formulation in various different packaging containers, temperature conditions, and with and without N₂. In this study, an unflavored sucrose-containing batch, PD137-30, was packaged into three alternate 4 ounce bottles—amber glass, amber PET, and white HDPE. The pH was 2.641.

All samples were manufactured and packaged under ambient conditions—no nitrogen was used. Samples were placed at 40 and 50° C. Samples were pulled after 20 days. The results of the analysis of packaging containers are presented in Table 1.

In addition, samples were placed in 20 mL vials and stored at 40° C., RT, refrigerator, and freezer for visual evaluation. Samples were also subjected to freeze/thaw and heat/cool cycle studies. After three months, no changes were seen in any samples, except for some slight discoloration at 40° C., most likely the result of sucrose carmelizing. Samples stored in the freezer did not freeze, nor were any precipitates present. The pH of these samples (results presented in Table 2) was also measured at the three-month time point.

TABLE 1
Stability results on sucrose-containing formulation after 20 days

| | | | % total |
|---|---|---|---|
| | | | degradation |
| Bottle | Storage Condition | Assay (% claim) | product* |
| Amber glass | 50 deg. C. | 99.9 | 1.3 |
| | 40 deg. C. | N/A | 1.6 |
| | RT (control) | 100.3 | 1.0 |
| PET | 50 deg. C. | 99.6 | 1.7 |
| | 40 deg. C. | N/A | 1.7 |
| | RT | N/A | 1.8 |
| HDPE | 50 deg. C. | 94.2 | 4.1 |
| | 40 deg. C. | N/A | 3.7 |
| | RT | N/A | 3.7 |

*Analysis of the degradation products on these samples was completed at the 7 month time point.

TABLE 2
pH measurement at 3 months [of PD137-30]

| Storage | | | Refrig. | Freezer | Freeze/ | Heat/ |
|---|---|---|---|---|---|---|
| Condition | RT | 40 deg. C. | 5 deg. C. | −20 deg. C. | Thaw | Cool |
| PH | 2.59 | 2.55* | 2.78 | 2.82* | 2.65 | 2.75* |

*Results are the average of 2 determinations.

A study was also conducted to compare the stability of the sucrose-containing formulation with and without nitrogen. For this study, a batch of the same formulation was made and packaged in a nitrogen tent. A portion of this batch was then removed from the nitrogen tent and mixed on the open bench for approximately 1 hour before packaging under ambient conditions. The samples were packaged in 22 mL glass headspace vials with caps crimped in place. These samples were then placed at room temperature, 40, 50, and 60° C. The results of this study indicate that loratadine is markedly more stable when packaged under nitrogen. In addition, the stability of the sodium benzoate was also slightly improved. The pH of the product tended to increase under all conditions. The presence of nitrogen had no impact on the pH change.

### EXAMPLE 2

Several sample batches were made with various ingredients for stability testing and characterization of degradation product. The compositions of these batches are shown in Table 3.

TABLE 3
Compositions of trial batches used to characterize degradation
product

| Lot # | Composition (Loratadine - 10 mg/10 mL) | pH |
|---|---|---|
| PD137-144 | DI Water, citric acid, glycerin, propylene glycol | 2.71 |
| PD137-146 | Same as PD137-144 plus flavor | 2.73 |
| PD137-147 | Same as PD137-144 plus sodium benzoate* | 2.74 |
| PD137-148 | DI Water, citric acid, glycerin, propylene glycol, | 2.63 |
| | sucrose | |

*Additional citric acid added to adjust pH.

These samples were placed in 4 oz. amber PET bottles and stored at 40 and 60° C. The batches were made under ambient conditions. The results at 8 and 12 weeks are shown in Table 4.

TABLE 4
Results of degradation products of sucrose-containing formulations

| | | 8 weeks | | 12 weeks | |
|---|---|---|---|---|---|
| | Storage | Assay | Degradation | Assay | Degradation |
| Lot # | Temp. | (% Claim) | Products | (% Claim) | Products |
| PD137- | 40 deg. C. | 103.5 | ND | 100.4 | ND |
| 144 | 60 deg. C. | 107.3 | ND | NT | NT |
| PD137- | 40 deg. C. | 105.1 | ND | 103.2 | ND |
| 146 | 60 deg. C. | 105.1 | ND | NT | NT |
| PD137- | 40 deg. C. | 102.9 | ND | 103.5 | ND |
| 147 | 60 deg. C. | 104.1 | 0.18% | NT | NT |
| PD137- | 40 deg. C. | 95.3 | 0.73% | 95.4 | 0.75% |
| 148 | 60 deg. C. | 98.9% | 0.40% | NT | NT |

ND = None Detected, NT = Not Tested.

In a follow-up study, batches were made with an alternate type of sucrose (lot #PD156-15) and sorbitol (lot #PD156-24). The alternate sucrose was C&H sucrose, which is claimed to be the purest sucrose available. As a control, a batch (lot #PD156-12) was made without any sucrose and only a small amount of additional water (theoretical loratadine concentration =278% of claim). Samples of each batch were packaged in 20 glass headspace vials. All batches were made under ambient conditions. The results of this study are shown in Table 5.

TABLE 5
Stability of sucrose-containing loratadine formulations

| | | 2 weeks | | 4 weeks | |
|---|---|---|---|---|---|
| | Storage | Assay | Degradation | Assay | Degradation |
| Lot # | Temp. | (% Claim) | Products | (% Claim) | Products |
| PD156- | RT | 268.6 | ND | NT | NT |
| 12 | 40 deg. C. | 268.4 | ND | NT | NT |
| Initial = | 50 deg. C. | 268.7 | ND | NT | NT |
| 269.7% | 60 deg. C. | 272.4 | ND | NT | NT |
| of claim | | | | | |
| PD156- | RT | 98.0 | ND | NT | NT |
| 15 | 40 deg. C. | 96.3 | 0.39% | 96.2 | 0.45% |
| Initial = | 50 deg. C. | 96.7 | 0.36% | NT | NT |
| 98.8% | 60 deg. C. | 96.6 | 0.37% | 96.4 | 0.39% |
| of claim | | | | | |
| PD156- | RT | 95.1 | ND | NT | NT |
| 24 | 40 deg. C. | 95.2 | ND | 93.8 | 0.09% |
| Initial = | 50 deg. C. | 93.6 | 0.11% | NT | NT |
| 95.4% | 60 deg. C. | 92.4 | 0.32% | 91.5 | 0.26% |
| of claim | | | | | |
| [clarify] | | | | | |

ND = None Detected, NT = Not Tested.

### EXAMPLE 3

Table 6 provides a comparison between the composition of an essentially sugar-free formulation according to the invention and a liquid loratadine formulation currently on the market.

TABLE 6

| Ingredient | Sucrose formulation | Sugar-free formulation |
|---|---|---|
| Sucrose Syrup #2 | 66% v/v | — |
| Hydroxypropyl | — | 0.45% w/v |
| Methylcellulose | | |
| Sodium Saccharin | — | 0.1% w/v |
| Sodium Benzoate | 0.1% w/v | 0.1% w/v |
| Citric Acid, anhydrous | 0.85% w/v | 0.85% w/v |
| Glycerin 96% | 9.0% w/v | 9.0% w/v |
| Flavor | 0.3% v/v | 0.3% v/v |
| Propylene Glycol | 9.1% v/v | 9.1% v/v |
| Loratadine | 0.1% w/v | 0.1% w/v |
| Deionized Water | qs ad 100 mL | qs ad 100 mL |

Samples of the above formulations were placed on stability in 20 mL headspace vials at 25, 40, and 60° C. The vials were filled such that the headspace to product volume ratio was about that of a 16 oz. bottle. The effect of nitrogen was also evaluated. Thus the sugar-containing formula was packaged under both nitrogen and ambient atmosphere. The results are presented in the following tables.

TABLE 7
Assay results (% of Claim) for loratadine at 25° C.

| | Sucrose Formula | Sucrose Formula | Sugar-free Formula |
|---|---|---|---|
| Weeks | 100% N2 | Ambient Air | Ambient Air |
| 0 (Initial) | 99.7 | 99.7 | 96.9 |
| 4 | Not tested | Not tested | 98.2 |
| 8 | 98.7 | 94.1 | 100.1 |
| 12 | 99.4 | 94 | 97.8 |

TABLE 8
Degradation Product level (% of active) at 25° C.

| | Sucrose Formula | Sucrose Formula | Sugar-free Formula |
|---|---|---|---|
| Weeks | 100% N2 | Ambient Air | Ambient Air |
| 0 | 0.3 | 0.3 | 0 |
| 4 | Not tested | Not tested | 0 |
| 8 | 0.1 | 0.6 | 0 |
| 12 | 0.3 | 1.1 | 0 |

TABLE 9
Assay results (% of Claim) for loratadine at 40° C.

| | Sucrose Formula | Sucrose Formula | Sugar-free Formula |
|---|---|---|---|
| Weeks | 100% N2 | Ambient Air | Ambient Air |
| 0 | 99.7 | 99.7 | 96.9 |
| 4 | 99.3 | 95.1 | 97.4 |
| 8 | 98.4 | 93.7 | 97.7 |
| 12 | 98.5 | 94.5 | 97.1 |

TABLE 10
Degradation Product level (% of active) at 40° C.

| | Sucrose Formula | Sucrose Formula | Sugar-free Formula |
|---|---|---|---|
| Weeks | 100% N2 | Ambient Air | Ambient Air |
| 0 | 0.3 | 0.3 | 0 |
| 4 | 0.3 | 0.8 | 0 |
| 8 | 0.1 | 0.5 | 0 |
| 12 | 0.4 | 0.9 | 0 |

TABLE 11
Assay results (% of Claim) for loratadine at 60° C.

| | Sucrose Formula | Sucrose Formula | Sugar-free Formula |
|---|---|---|---|
| Weeks | 100% N2 | Ambient Air | Ambient Air |
| 0 | 99.7 | 99.7 | 96.9 |
| 4 | 97.7 | 94.9 | 96.1 |
| 8 | 96.2 | 93 | 96.1 |
| 12 | 94.5 | 92.2 | 93.2 |

TABLE 12
Degradation Product level (% of active) at 60° C.

| | Sucrose Formula | Sucrose Formula | Sugar-free Formula |
|---|---|---|---|
| Weeks | 100% N2 | Ambient Air | Ambient Air |
| 0 | 0.3 | 0.3 | 0 |
| 4 | 0.3 | 1.1 | 0.1 |
| 8 | 0.1 | 0.3 | 0.1 |
| 12 | 0.3 | 1 | 0 |

Based on the results for potency, the sucrose-containing formula packaged under nitrogen and the sugar-free formula under ambient conditions are comparable. Both products are superior to the sucrose formula packaged under ambient conditions. Based on the results for degradation products, however, the sugar-free formula packaged under ambient conditions is superior to the sucrose formula packaged under nitrogen, which in turn is superior to the sucrose formula packaged under ambient conditions. These results indicate a significant and surprising enhancement of storage stability is achieved by the essentially sugar free formulations.

The above description and examples are only illustrative of preferred embodiments which achieve the objects, features, and advantages of the present invention, and it is not intended that the present invention be limited thereto.

What is claimed as new and desired to be protected by Letters Patent of the United States is:

1. A storage stable, essentially sugar-free, oral pharmaceutical composition, comprising a therapeutically effective amount of a piperidine antihistamine, a viscosity imparting agent, a preservative, a buffer to control pH to about 2 to about 4, and water, wherein said composition does not contain an aminopolycarboxylic acid.

2. The pharmaceutical composition of claim 1, wherein said antihistamine is selected from the group consisting of loratadine, descarboethoxyloratadine, and azatadine.

3. The pharmaceutical composition of claim 2, wherein the viscosity-imparting agent is selected from the group consisting of hydroxyethyl cellulose, methyl cellulose, sodium carboxymethyl cellulose, microcrystalline cellulose, hydroxypropyl cellulose, hydroxypropyl methylcellulose, gelatin, polyethylene glycol, and a water-soluble carboxyvinyl polymer.

4. The pharmaceutical composition of claim 1, further comprising a sweetener.

5. The pharmaceutical composition of claim 4, wherein the sweetener is sodium saccharin.

6. The pharmaceutical composition of claim 4, wherein the sweetener is selected from the group consisting of malitol and sorbitol.

7. The pharmaceutical composition of claim 1, wherein the preservative is selected from the group consisting of parabens, propyl-p-hydroxybenzoates, sorbic acid, and sodium benzoate.

8. The pharmaceutical composition of claim 1, wherein the preservative is selected from the group consisting of methylparaben, butylparaben, and propylparaben.

9. The pharmaceutical composition of claim 1, wherein the buffer is selected from the group consisting of tartaric acid and citric acid.

10. The pharmaceutical composition of claim 1, further comprising a co-solvent.

11. The pharmaceutical composition of claim 1, further comprising a co-solvent selected from the group consisting of glycerin, polyethylene glycol, ethyl alcohol, and propylene glycol.

12. The pharmaceutical composition of claim 1, further comprising a flavoring agent.

13. The pharmaceutical composition of claim 1, wherein the composition comprises a degradation product in an amount up to about 0.1% of the antihistamine after 8 weeks at 60° C.

14. The pharmaceutical composition of claim 13, wherein the composition comprises a degradation product in an amount of up to about 0.1% after 12 weeks at 60° C.

15. The pharmaceutical composition of claim 1, further comprising a therapeutically effective amount of a pharmaceutically active compound selected from the group consisting of a decongestant, an analgesic, an antitussive, and an expectorant.

16. A pharmaceutical composition comprising 76 ml purified water, 0.450 g hydroxypropyl methylcellulose, sodium saccharin, 0.1 g sodium benzoate, 0.85 citric acid, 9 ml glycerin, 9.1 ml propylene glycol, and 0.1 g loratadine, wherein said composition does not contain an aminopolycarboxylic acid.

17. A method of treating allergic reactions in a mammal comprising administering to said mammal an anti-allergic effective amount of a pharmaceutical composition as defined in claim 1.

18. The method of claim 17, wherein the mammal is a human.

19. The method of claim 17, wherein the administration is oral.

20. The method of claim 17, wherein the antihistamine is loratadine.

21. The method of claim 17, wherein the allergic reaction is seasonal allergic rhinitis.

22. A method of treating mental disorders in a mammal comprising administering an effective amount of a pharmaceutical composition as defined in claim 1.

23. The method of claim 22, wherein the mental disorder is selected from the group consisting of depression, alcoholism, weight management disorders, social disorder, impotent/sexual dysfunction, panic, and obsessive/compulsive disorder.

24. A method of treating vascular disorders in a mammal comprising administering an effective amount of a pharmaceutical composition as defined in claim 1.

25. The method of claim 24, wherein the vascular disorder is selected from the group consisting of migraines, stroke, orthostatic hypotension, gastrointestinal stasis, nausea, dizziness, and jet lag.
